(19) United States
(12) Patent Application Publication
(10) Pub. No.: US 2009/0187082 A1
(43) Pub. Date: Jul. 23, 2009

(54) SYSTEMS AND METHODS FOR DIAGNOSING THE CAUSE OF TREND SHIFTS IN HOME HEALTH DATA

(76) Inventors: Paul E. Cuddihy, Ballston Lake, NY (US); Mark D. Osborn, Clifton Park, NY (US)

Correspondence Address: GENERAL ELECTRIC COMPANY;GLOBAL RESEARCH, PATENT DOCKET RM. BLDG. K1-4A59, NISKAYUNA, NY, 12309, US

(21) Appl. No.: 12/017,185

(22) Filed: Jan. 21, 2008

(51) Int. Cl. A61B 5/00

(52) U.S. Cl. 600/300

(57) ABSTRACT

A system and method for determining the cause of a trend shift in physiological data received from a patient under observation includes receiving physiological data on a plurality of measured physiological parameters from the patient and performing a statistical analysis on a portion of the physiological data to determine a measured shift over a confidence interval in each of the plurality of physiological parameters. A signature shift is defined for each of the plurality of physiological parameters that is indicative of a pre-determined medical condition and the measured shift confidence interval of each of the plurality of physiological parameters is compared to these signature shifts. From this comparison between the measured shift confidence interval and the signature shift of each of the plurality of physiological parameters, a physiological assessment is formulated.

### BACKGROUND OF THE INVENTION

The present invention relates generally to automated statistical systems and methods. More specifically, the present invention relates to a system and method for identifying an underlying change in physiological processes that may be indicative of a problem manifested ultimately in a disease or condition by way of detecting trend shifts in physiological data.

Patient health monitoring provides assessments on the ongoing condition of a patient by way of physiological data gathered on-site with the patient, the data being gathered either in a healthcare facility or via at home patient monitoring. This data, which typically is comprised of physiological parameters such as heart rate, blood pressure, weight, and blood oxygen levels, is acquired and transmitted to a processing unit for subsequent analysis. The data acquired is typically analyzed in an automated fashion and feedback is provided to a healthcare provider.

For particular ailments such as diabetes, hypertension, and congestive heart failure, the condition of a patient can change rapidly. Thus, it is important for patient monitoring systems to be able to detect and measure shifts and/or variances in physiological data that may be indicative of a potential change in a patient's condition and of problems that may be likely to develop in a patient, before such a potential problem actually develops to a serious state. A clinician must examine shifts in data for each physiological parameter being measured. Depending on whether the shifts for each parameter are examined individually or in combination with one another, different inferences may be drawn by a clinician as to the cause for the shifts. That is, the shifting of various physiological parameters, in relation to one another, may be indicative of a certain medical condition. Such parameter shifts may be sudden or occur over time, but in each case, these shifts can be indicative of a certain medical condition when examined together.

To aid in diagnosis of a patient based on measured physiological parameters, automated diagnostic systems have been introduced in the art. Existing automated monitoring systems have typically been designed to implement a scoring technique to determine a patient condition. In an automated scoring system, each patient is scored on the basis of various measured physiological parameters and the patient's score is determined by adding up the point total. The combined score obtained from the different physiological parameters reflects a certain risk level associated with a patient. While useful in determining an at-risk status for a patient, the resulting score provides no diagnosis of the cause for the measured physiological state or for trend shifts detected in the physiological data. In fact, no such diagnosis would even be possible in prior art scoring systems, as the automated scoring system merely looks at each physiological parameter independently, without regard to interactions between the parameters.

Outside the art of medical diagnosis, systems have been designed that are capable of detecting and diagnosing the cause of trend shifts in performance data associated with mechanical, electrical, and electro-mechanical systems are known in the art. Such diagnostic systems are typically configured as data-driven systems and/or rule-based systems. Data-driven systems, also referred to herein as “case-based systems” or “experience-based systems,” require the incorporation of a relatively large number of examples or validation cases before a given diagnostic system “learns” how to make an accurate diagnosis. Such diagnostic systems are prone to over-fitting data and making important decisions based on infrequent and/or irrelevant information. These diagnostic systems, however, are useful for diagnosing problems where examples or validation cases are plentiful and there is relatively little domain knowledge.

For rule-based systems, conversely, examples or validation cases are not plentiful. In such a domain, experts typically prefer to write rules explaining what they hope to find and how to make diagnoses. These manually written rules suffer from the fact that they do not always match the examples or validation cases perfectly. Differences in the way symptoms are measured and the inability to predict the magnitude and/or speed of symptoms cause the rules to be imprecise, even if they are relatively easily interpreted and corrected by those performing manual diagnoses. An automated diagnostic system performing such diagnoses, such as a computerized diagnostic system, has a relatively difficult time correcting the rules in real time.

Additionally, when multiple parameters are examined over time, rule-based systems, also referred to herein as “model-based systems,” suffer from model uncertainty (related to the inability to determine how large of a trend shift to correlate to a given problem) and measurement uncertainty (related to the inability to determine the extent of the effect of noise on a given trend shift). Multiple parameters must, however, be considered in order to make an accurate diagnosis. Typically, these problems have been addressed via thresholding and the use of trend shift alerts. These trend shift alerts often utilize dimensionality that is too low to make an accurate diagnosis and, historically, rules are only corrected when they fail, i.e., they are not optimized.

Historically, such diagnostic systems used for diagnosing the cause of trend shifts of performance data have been limited to use with mechanical, electrical, or electro-mechanical systems, and have not been applied to the field of medical patient diagnosis. The reasons for this are many. First, the human body is a dynamic system exhibiting highly complex behavior, in which medical cause-effect relationships, the relations between diagnoses and their symptoms, are hardly ever one-to-one. Differentiation of diagnoses that share an overlapping range of symptoms is therefore inherently difficult. Secondly, the body's current state is almost never sufficiently described by the instantaneous values of its observable parameters or any time-ignorant derivation thereof. However, the observations/data necessary for detecting trend shifts and formulating a diagnosis as the cause of these shifts can often not be made on a continuous basis in the field of patient health monitoring. To the contrary, because many diagnostically meaningful observations can only be obtained at rather high risk to the patient or at a very high cost, one would have to make do with significantly less than desirable information when formulating a diagnosis. This is especially a problem for the diagnosis of dynamic perturbations that evolve over an extended period of time, in which gapless recording of the time course of physiologically decisive parameters is desired. Additionally, issues of pre-existing medical conditions in a patient and of prescribed treatments that are in place at the time of patient monitoring must be taken into account when detecting trend shifts in physiological parameters and formulating a diagnosis therefrom. A diagnostic monitor must therefore be aware of the medical history of the monitored subject.

Although taken alone none of the issues set forth above may be unique to the medical domain, taken together they add to an intricacy surpassing that of existing diagnostic systems in place for diagnosing trend shifts. Therefore, a need exists for a system and method that allows for the detection and measurement of trends in acquired physiological data. A need also exists for models against which the shifts in physiological data can be compared for purposes of identifying an underlying change in physiological processes that may be indicative of a problem manifested ultimately in a disease or condition. A need further exists for a system and method that allows for the entering examples or validation cases against which the models may be evaluated and optimized.

### BRIEF DESCRIPTION OF THE INVENTION

Embodiments of the invention provide a system and method that allows a clinician to enter one or more fuzzy functions related to one or more trend shifts into an automated diagnostic system. The automated diagnostic system then uses rules from the fuzzy functions to diagnose possible physiological conditions associated with a patient. A plurality of examples or validation cases is used to improve and refine the one or more fuzzy functions, optimizing the performance of the automated diagnostic system. Preferably, the improved fuzzy functions are presented to a clinician for verification.

In accordance with one aspect of the invention, an automated method for diagnosing the cause of a trend shift in physiological data includes the step of receiving physiological data from a patient under observation, the physiological data comprising data on a plurality of measured physiological parameters. The method also includes the steps of performing a statistical analysis on a portion of the physiological data to determine a measured shift confidence interval in each of the plurality of physiological parameters and defining a signature shift for each of the plurality of physiological parameters, wherein the signature shifts for the plurality of physiological parameters are indicative of a pre-determined medical condition. The method further includes the steps of comparing the measured shift confidence interval of each of the plurality of physiological parameters to the signature shift associated with each of the plurality of physiological parameters and detecting a change in patient condition based on the comparison between the measured shift confidence interval and the signature shift of each of the plurality of physiological parameters.

In accordance with another aspect of the invention, a patient monitoring system includes a patient monitoring device configured to acquire physiological data from a patient under observation, the physiological data providing a measurement of at least one physiological parameter. The patient monitoring system also includes a computer in communication with the patient monitoring device to receive physiological data therefrom. The computer is programmed to receive physiological data from the monitoring device and select an analysis period that includes at least a portion of the acquired physiological data, the analysis period having a start date and an end date. The computer is further programmed to select data sets from the analysis period near the start date and the end date that have a predetermined size without violating normal scatter, measure a shift confidence interval between the data set near the start date and the data set near the end date using one or more statistical tests, and combine the shift confidence interval with a fuzzy model to achieve a physiological condition assessment, the fuzzy model describing how the mean associated with the at least one physiological parameter shifts when a predetermined physiological condition is present.

In accordance with yet another aspect of the invention, a computer readable storage medium includes thereon a computer program to provide a physiological condition assessment based on trend shifts in physiological data. The computer program comprises a set of instructions that, when executed by a computer, causes the computer to receive physiological data on a plurality of physiological parameters, determine a trend shift in the plurality of physiological parameters based on a statistical analysis of the physiological data, input the trend shift into a fuzzy model to identify a patient condition, and validate the fuzzy model using a plurality of validation cases comprising examples of diagnosed medical conditions determined from known shifts in the plurality of physiological parameters. The set of instructions further causes the computer to use an evaluation function to determine how well the fuzzy model differentiates the identified patient condition from a plurality of incorrect patient conditions for the plurality of validation cases.

These and other advantages and features will be more readily understood from the following detailed description of preferred embodiments of the invention that is provided in connection with the accompanying drawings.

### BRIEF DESCRIPTION OF THE DRAWINGS

The drawings illustrate embodiments presently contemplated for carrying out the invention.

In the drawings:

FIG. 1 is a schematic block diagram of a diagnostic patient monitoring system incorporating the present invention.

FIG. 2 is a series of plots illustrating a sample data set for three parameters collected over a period of several months and utilized by the diagnostic systems and methods of the present invention.

FIG. 3 is a series of plots illustrating how two rules associated with the diagnostic systems and methods of the present invention function.

FIG. 4 is a series of plots illustrating several measurement steps associated with the diagnostic systems and methods of the present invention.

FIG. 5 is a graph illustrating a method for evaluating a fuzzy function associated with the diagnostic systems and methods of the present invention.

FIG. 6 is a plot a sample data set for patient weight collected over a period of several months and utilized by the diagnostic systems and methods of the present invention.

FIG. 7 is a plot of a sample data set for diastolic blood pressure, systolic blood pressure, and pulse collected over a period of several months and utilized by the diagnostic systems and methods of the present invention.

FIG. 8 is a plot of a sample data set for gross motor activity collected over a period of several months and utilized by the diagnostic systems and methods of the present invention.

FIG. 9 is a plot illustrating elbow identification and outlier removal for weight data associated with the diagnostic systems and methods of the present invention

### DETAILED DESCRIPTION OF THE PREFERRED EMBODIMENT

As an overview, the automated diagnostic systems and methods of the present invention allow a medical expert to build a plurality of fuzzy models describing how the mean of each of a plurality of physiological parameters shifts for each of a plurality of predetermined medical conditions. Outliers are removed using standard statistical techniques or domain-specific rules. The physiological data is split by time in such a way as to allow linear or non-linear regressions to be run through each of a plurality of physiological data segments with the lowest total residuals. The split points are evaluated to determine which are most likely to represent the beginning of a given problem. Data sets from the end of the physiological data and around the best split point(s) are chosen such that each data set includes as many data points as possible without a standard deviation too far above that of the standard deviation of the linear regression residuals of the entire data window. T-tests or the like are then run on the data sets and rolled together with the fuzzy functions incorporated in the fuzzy models. Modeled problems that receive the highest scores are reported.

Advantageously, the models of the trend shifts mapped to each problem are relatively simple to understand and maintain and effectively capture the knowledge of a medical expert. T-tests or the like and fuzzy functions combine the uncertainty in the physiological data and model more effectively than the human eye/brain. The diagnostic systems and methods of the present invention require little or no human interaction and may be delivered, for example, via the Internet.

In an extension of the present invention, genetic algorithms and six-sigma techniques are used to combine experience-based and rule-based diagnostics in order to enjoy the benefits of both, further increasing accuracy of trend shifts and their association to a diagnosed medical condition. An evaluation function is used to determine how well a given set of rules differentiates correct and incorrect physiological parameter assessments (i.e., patient conditions) for a set of examples or validation cases. The output of the tool is a list of confidence values, 0 to 1, for each of a plurality of identified patient conditions. There is a fuzzy function for each dimension of the rule. Predetermined fuzzy rules are changed randomly within established guidelines and the evaluation function is recalculated. Using known genetic algorithms, deviations that cause better results may be combined and reevaluated. Preferably, this process continues until no better solution may be found. A healthcare provider or the like then validates these results in order to ensure that no over-fitting has occurred.

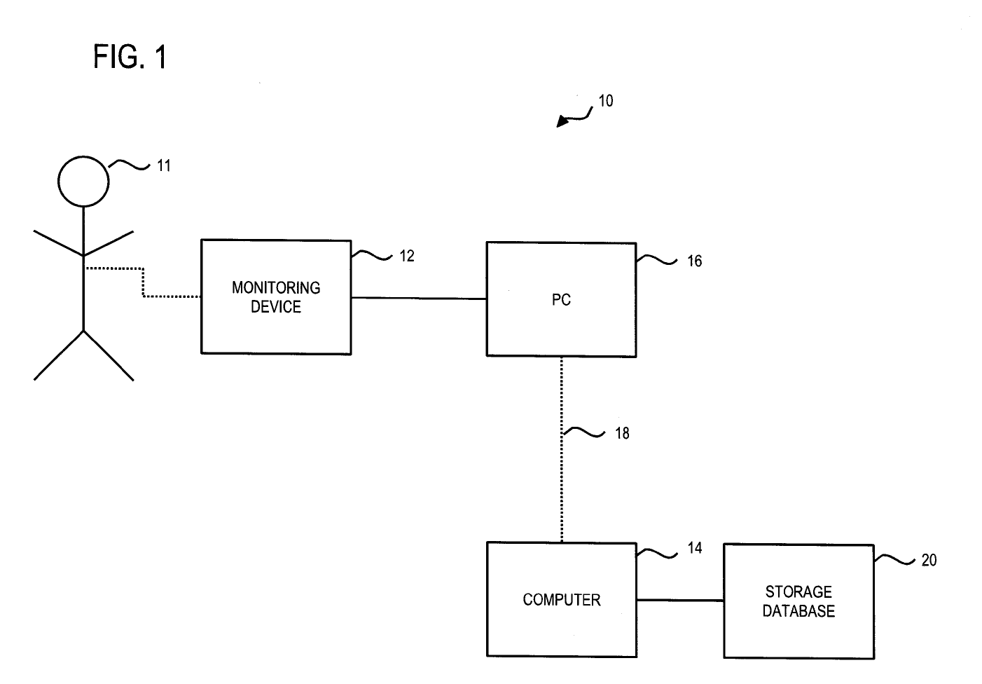

Referring to FIG. 1, a block diagram shows one embodiment of a patient monitoring system 10 for use with the present invention. Patient monitoring system 10 includes a monitoring device 12 that is located on-site with a patient to be monitored. Monitoring device 12 can be configured to automatically measure physiological parameters of the patient, or alternatively, be in the form of a device that allows for the patient to perform manual self-check tests. While shown as a single device, monitoring device 12 can encompass a plurality of devices, each of which measures a specific physiological parameter. The physiological parameter(s) acquired by monitoring device(s) can include, but are not limited to, blood pressure, weight, pulse, and saturation of peripheral oxygen (Sp_{O2}).

The monitoring device 12, either in an automatic or manual fashion, acquires physiological data from the patient. The physiological data is stored within the device by way of a short-term, volatile memory and then is transmitted therefrom to a remotely located computer 14 or processing/server system located at a healthcare facility for further evaluation. Transmission of the physiological data can occur via any of a plurality of well-known methods. That is, data may be transferred from monitoring device 12 to a personal computer 16 located on-site with the patient and interfaced with the monitoring device. The PC 16 can then transfer the acquired physiological data to computer and/or server system 14 located at a designated healthcare facility via a communications medium 18 such as the Internet or telephone networks. Alternatively, the monitoring device 12 can be configured to send data directly therefrom, or a home hub may be utilized. Regardless of the exact manner of transfer of physiological data, the physiological data received by computer 14 at the healthcare facility is then stored in an electronic database 20 containing a patient profile.

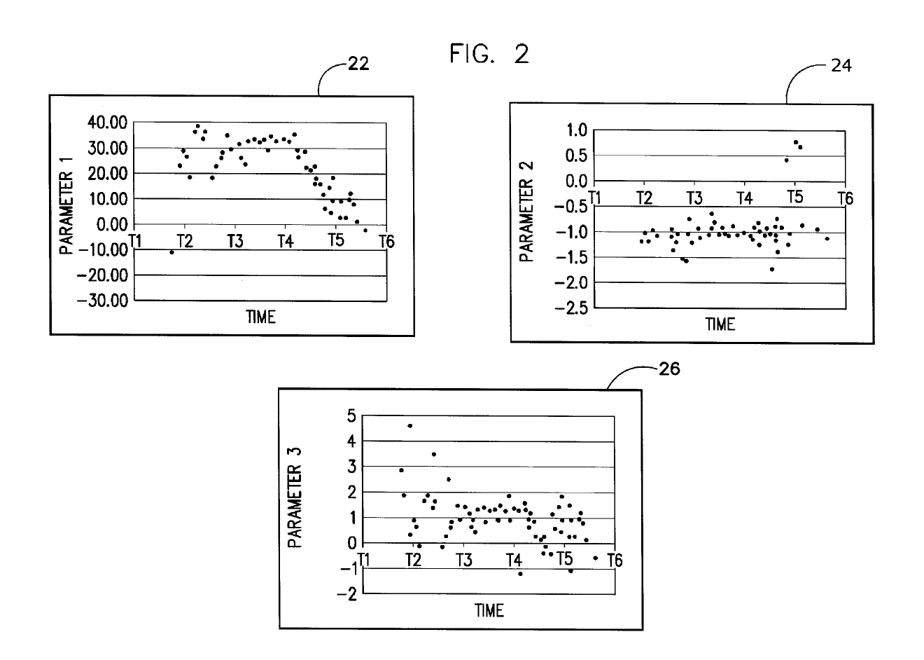

The patient monitoring system, 10 by way of computer 14 is designed to measure trend shifts in the physiological data for a plurality of physiological parameters, parameter 1, parameter 2, . . . , and parameter n. FIG. 2 illustrates a sample data set for three physiological parameters, parameter 1 22, parameter 2 24, and parameter 3 26, collected over a period of several months. These parameters can include, but are not limited to, blood pressure, weight, pulse, and saturation of peripheral oxygen (SpO₂).

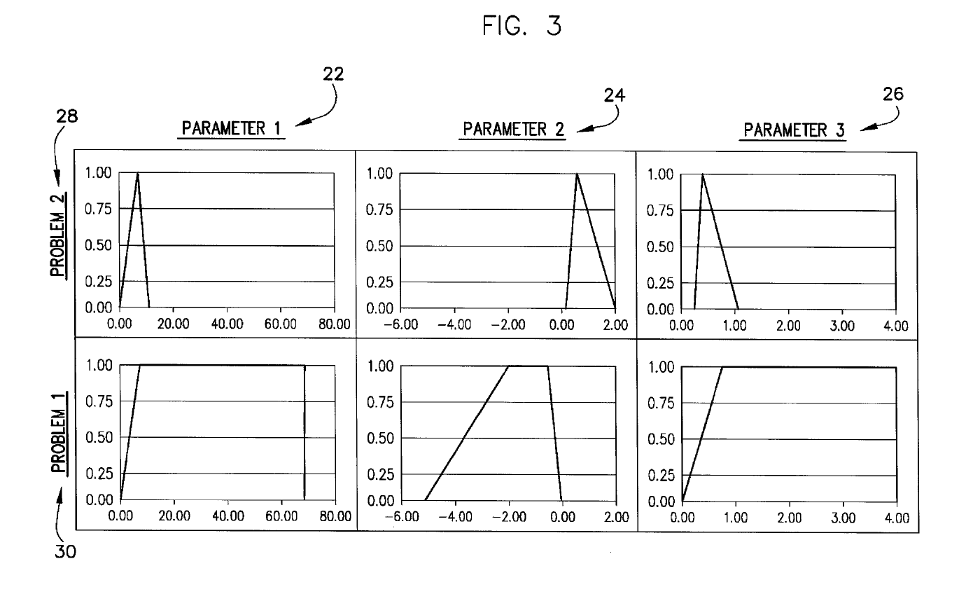

The shifts in data for a plurality of physiological parameters can be examined in combination to detect a change in condition of the patient. That is, computer 14 (shown in FIG. 1) is programmed to function as a “diagnostic system” configured to perform a physiological condition assessment based on trend shifts in data associated with the physiological parameters for purposes of identifying a specific medical condition. Assessments are made based upon trend shifts, and trends in three or four parameters up or down yield several dozen rules (2⁴=16, 3⁴=81 for up/down/no change). Each detected change in patient condition is associated with a signature shift for each physiological parameter being measured. For a rule to match, measured shifts must match the signature shift for each physiological parameter. FIG. 3 illustrates how two rules associated with the diagnostic system function. The first rule 28 looks for about 5 degrees shift related to parameter 1 22, about 1.0% shift related to parameter 2 24, and about 0.5% shift related to parameter 3 26. The second rule 30 looks for about 7 degrees shift up related to parameter 1 22, about 0.5% to about 2.0% shift down related to parameter 2 24, and about 0.5% shift up related to parameter 3 26. In FIG. 3, the horizontal axes represent the amount of parameter shift and the vertical axes represent the confidence of the corresponding physiological condition assessment. In one embodiment of the invention, an alert is generated by system 10 when the confidence level meets a pre-determined confidence threshold. Such an alert can thus notify a clinician that a physiological condition assessment of the patient has been successfully generated. In addition to shift amounts, the diagnostic system uses the duration of the shift as an extra parameter that behaves differently from the other parameters. Identical shifts may be differentiated based on the period of time over which they occur.

Advantageously, the diagnostic system acts independently of a fixed window in which shifts are measured, but instead analyzes data acquired during an analysis period or short-term window. That is, an analysis period in which physiological data is analyzed can be determined by a clinician based on a specified criteria (i.e., start date of a treatment and/or therapy, start date of patient monitoring, etc.) or based upon a statistical analysis of the acquired physiological data. In one embodiment, the patient monitoring system 10 determines the start date of the most recent shift based on identification of elbows/split points in the physiological data. The elbows/split points are evaluated to determine a period most likely to represent the beginning of a given problem, as measured across all parameters, and shifts are measured only during that period. The diagnostic system of the present invention also incorporates one or more algorithms that combine noise in the physiological data (as measured using a t-test statistic) with the noise in the model (as represented by the fuzzy functions). This allows for the accurate ranking of possible causes for a change in patient condition.

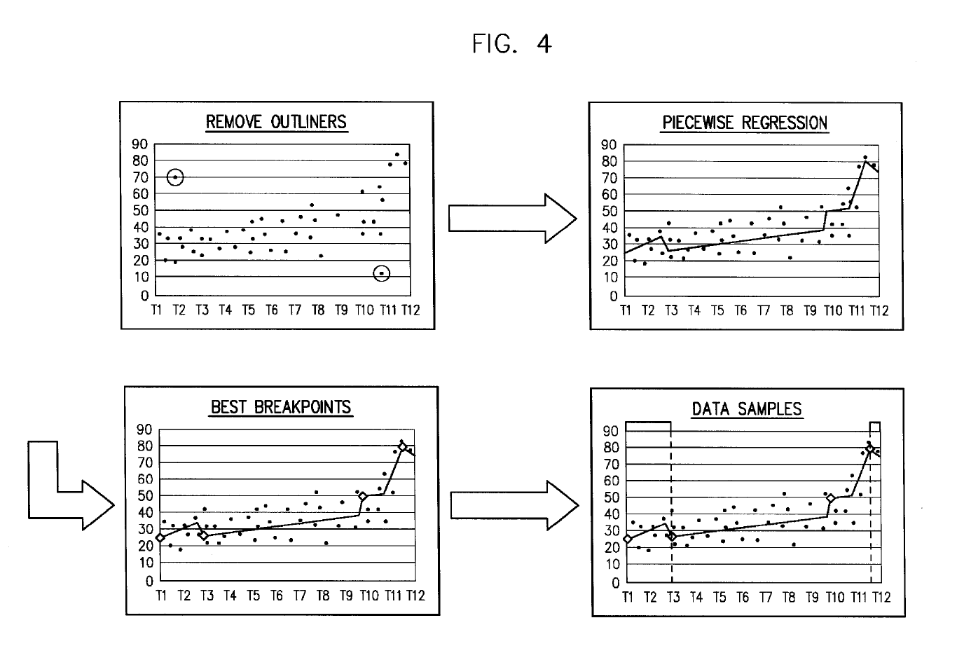

In one embodiment, the patient monitoring system incorporates the following measurement steps, several of which are illustrated in FIG. 4: removing outliers by calculating a local standard deviation minus the point in question and removing the point in question if it falls outside of a specified z (e.g., performing a control chart analysis); finding the best set of piecewise linear or non-linear regressions for a selected time range, the selected time range having a start date and end date determined by a clinician or by the identification of corner points that provide the largest shift; finding the local standard deviations representing normal scatter; picking data sets near the start date and end date that are as large as possible without violating normal scatter; using two sample t-tests to measure the mean shift between samples; determining a confidence interval around each mean shift; and combining these results with the fuzzy models to achieve a diagnosis.

Data sets are defined around the start date and the current date, and the confidence intervals around their means are compared using two sample t-tests. This reduces inconsistency and the manual estimation typically associated with trend shift measurement.

The output of the diagnostic system comprises an ordered list of rule matches. A complete list of rules is presented as several of the most likely diagnoses should be considered and it is useful to consider which diagnoses are least likely.

As shown in FIG. 4, outlier removal is performed on acquired patient data. The noise filtering or outlier removal associated with the diagnostic system should be appropriate for the domain involved and may include, for example, a two-pass process or the like as is well known to those of ordinary skill in the art.

Once outliers are removed, a piecewise linear regression algorithm is applied to the data for each physiological parameter. Alternatively, a non-linear regression algorithm may be applied, where appropriate. For every point except for the points at the beginning and end of the sample, one regression is fitted for all earlier data and another regression is fitted for all later data. The error is squared and recorded. The splitting point producing the lowest error is retained. This process is applied to each side recursively to each sub-section as long as it still has a predetermined number of points in it and covers at least a predetermined number of days (or years, months, weeks, hours, minutes, seconds, etc.). In order to catch newly developing shifts, the final line segment is split one additional time. While described above as a linear regression, it is also envisioned that any kind of curve can be fit to all the data since the last clinical review, or since the last clinical event.

For evaluating possible start dates and measuring shifts, a one of several methods for selecting data samples can be utilized. In one embodiment, and as shown in FIG. 4, a short-term standard deviation is calculated for the data. This involves calculating the distance of each point from the fitted curve and calculating the standard deviation of all of these values. The result represents the standard deviation of the short-term noise. To select a data set representing a potential start date, data points before the target point are evaluated, starting with a minimum data set size. The data set is expanded backwards as long as its standard deviation does not exceed a predetermined maximum noise ratio times the short-term noise standard deviation and the data set size does not exceed a maximum set, which will depend on the frequency with which physiological data is acquired. The function attempts to get a predetermined number of points, but makes adjustments as necessary to keep the maximum between two potential numbers of total points. The result is a data set large enough that is has scatter representative of the complete data set, but small enough to minimize the apparent scatter caused by trend shifts.

In another embodiment, and as mentioned above, it is envisioned that an analysis period in which physiological data is analyzed can be pre-determined by a clinician based on a specified criteria (i.e., start date of a treatment and/or therapy, start date of patient monitoring, etc.). The clinician can enter the desired analysis period as input into the patient monitoring system 10.

Once data sets from the start and end dates of the selected/statistically determined analysis period have been identified, statistical analysis on those data sets (and the physiological data contained therein) are performed to determine a measured shift in each measured physiological parameter. More specifically, statistical analysis on a confidence interval about the measured shift is performed. In one example, a t-test (e.g., two-sample t-test) is performed on the measured shift confidence interval.

Upon acquiring data on a measured shift for a physiological parameter over a specified confidence interval, that data is input into a fuzzy model that comprises a plurality of fuzzy rules. Each fuzzy rule is stored as a set of, for example, 4X values. These values represent the X's in increasing order where Y is [0,1,1,0]. Referring to Table 1, the diagnosis 4 rule expects a parameter 1 shift of between −3 and −1. Values between −10 and 0 are considered partial matches.

TABLE 1
Exemplary set of Fuzzy Rules Associated With Parameter 1

Parameter 1

| Diagnosis Rule | Fuz 0 | Fuz 1 | Fuz 1′ | Fuz 0′ |
|---|---|---|---|---|
| Diagnosis 1 Rule | 205 | 88 | 159 | 68 |
| Diagnosis 2 Rule | 240 | 181 | 209 | 90 |
| Diagnosis 3 Rule | 192 | 58 | 99 | 54 |
| Diagnosis 4 Rule | 8.17 | 15.27 | 18.87 | 22.12 |
| Diagnosis 5 Rule | 48 | 64 | 64 | 68 |

Functions are not evaluated against a single value shift estimate, but rather against the entire confidence interval of the shift calculation. Thus, a triangle function such as [5,10,10,15]—which represents the desire to match a shift of 10 degrees—may never evaluate to 1.0 unless there is no scatter in the data. Preferably, the fuzzy functions have plateaus that cover a reasonable noise band.

It should also be noted that some of the functions might be built with arbitrarily large values on one end of the function, such as [0,15,100,inf], where inf represents positive infinity. This is meant to cover any shift above 15 degrees (again, due to noise, the function will not evaluate to 1.0 until the shift is greater than 15 degrees).

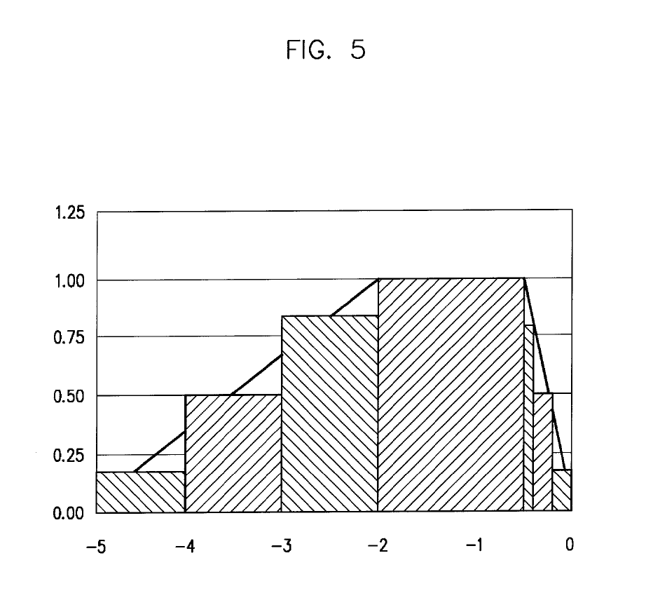

Each individual fuzzy function represents an expected mean shift. Given two data sets, a function is evaluated by simplifying the slopes into step functions, using two-sample t-tests or the like to evaluate the probability for each step, and summing the results. This is akin to integration and is illustrated in FIG. 5. FIG. 5 indicates, based on the fuzzy function represented, that clinician was looking for a mean shift of between about −0.5 and −2.0, but was willing to accept some match between about 0 and −5.0. The fuzzy function is represented as a step function. Each area is assigned a multiplier equal to the average value of the fuzzy function over its range. A two-sample t-test or the like is performed over the range of each area. The final probability is the sum of the t-test results times the multiplier for each step:

prob=sum(1,n)(ttest(sample1,sample2,n_{low},n_{high}))*(fuzzy(n_{low})+fuzzy(n_{high}))/2 [Eqn. 1]

Higher values for n provide more accurate results. For example, n may have a value of 5. The plateaus may be evaluated and the slopes split into two pieces each.

Scores for each individual rule are computed as described above. A score for duration is calculated by simply mapping the duration (end date minus start date) to the duration fuzzy function. A verification display may be made available to the clinician in the form of a rule screen showing the best matching rules. The display may show all of the parameters associated with a given rule in one row. For each parameter, the fuzzy function is displayed, representing the “expected” mean shift. The calculated mean shift is also displayed, with the confidence band used in the fuzzy calculation being shown. Preferably, the match value is also shown below each parameter.

### Example 1

The following example describes patient monitoring of heart failure patients and detection of trend shifts for a plurality of physiological parameters. The described simulation measures the physiological parameters of weight, systolic and diastolic blood pressure, pulse, and gross motor activity. Trend shifts are detected in each of the measured physiological parameters and input into a fuzzy model.

In the measured patients, weight is typically measured once per day. In heart failure patients considerable weight gain over a short period of time can be experienced due to peripheral edema in which the tissues swell due to fluid retention. For example, weight gain of (i) 2-3 lbs overnight and 3-5 lbs over a period of 5 days; or (ii) 3-4 lbs in one day and 5-6 lbs over a two-day period, is not uncommon. However, there is also the potential for a variance of up to 6 lbs per day in a stable patient due to normal intake and retention of fluids and solids depending on the time of day of the weight measurement being taken.

Blood pressure measurements are taken for a patient and include both the systolic (normal range between 90 and 135 mm Hg) and diastolic (normal range between 50 and 90 mm Hg) blood pressure. In a heart failure patient these values may typically range from anywhere between 140-150 for systolic and 95-105 for diastolic.

Pulse is measured for a patient using a digital blood pressure monitor. A normal resting heart rate is considered to be between 60-100 beats per minute.

Gross motor activity, is measured through the use of an accelerometer having a sensitivity on the order of >0.01-0.005 g worn on the non-dominant arm of the patient. The data is collected on a continual basis and recorded as the mean number of activities in a specified time interval (e.g., activities in most active 0.5 hr per day, M0.5).

Table 2 displays an example set of patient data recorded over a period of 296 days that provides the following statistics:

TABLE 2

| | | | BP - | | |
|---|---|---|---|---|---|
| | | BP - | Systolic | Pulse | Activity (activities |
| | Weight | Diastolic | (mm | (beats/ | in most active 0.5 hr |
| | (lbs) | (mm Hg) | Hg) | min) | per day, M0.5) |
| Mean | 205 | 88 | 159 | 68 | 35335 |
| Maximum | 240 | 181 | 209 | 90 | 107104 |
| Minimum | 192 | 58 | 99 | 54 | 38574 |
| Standard | 8.17 | 15.27 | 18.87 | 22.12 | 18161 |
| Deviation | | | | | |
| % | 48 | 64 | 64 | 68 | 27 |
| Missing | | | | | |

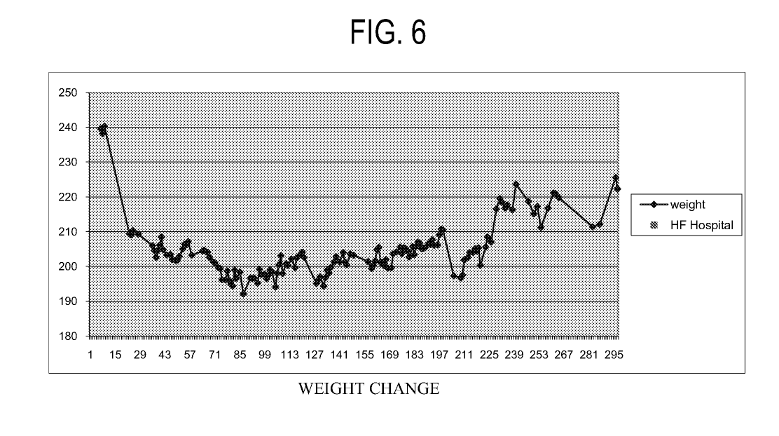

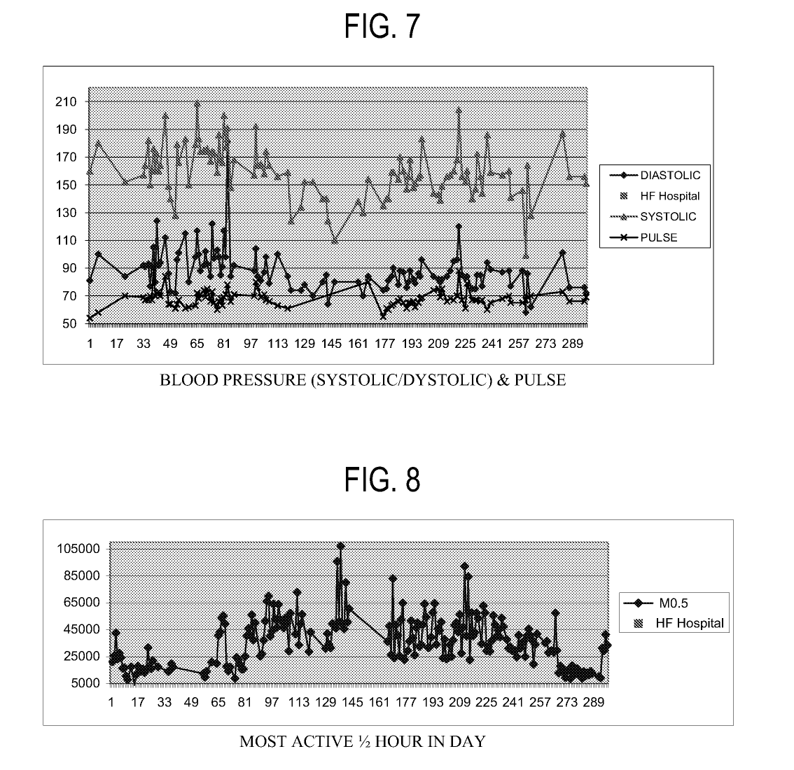

The patient data recorded over a period of 296 days is plotted in FIGS. 6-8.

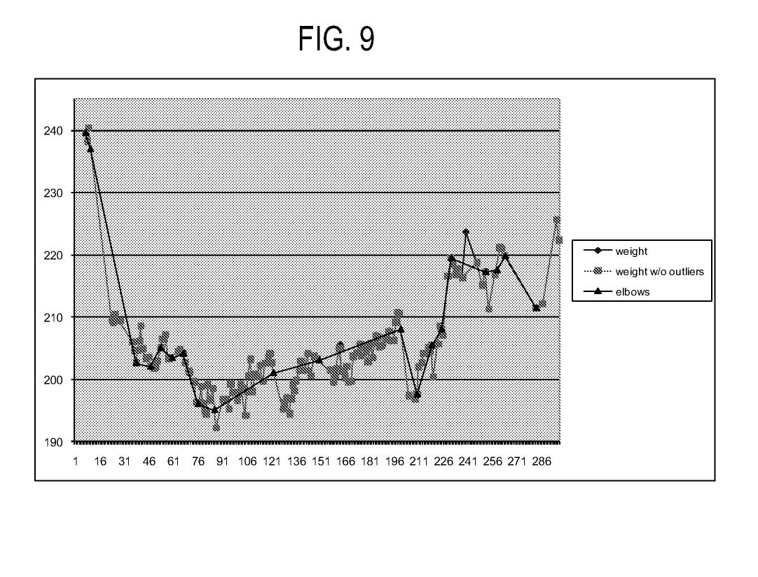

For each measured physiological parameter, trend shifts are detected using statistical analysis (e.g., two-step t-test). Thus, for each parameter, outliers are identified and new values for outlier data are substituted in. Elbows in the physiological data are identified, along with data sets around each elbow. For the plurality of parameters, data sets in each parameter are identified for other parameters' elbows. Data sets for these additional elbows are also identified and the most significant shift is determined based on a super-set of data sets. FIG. 9 displays both outlier removal and elbow identification for the patient's weight data. Additionally, from the data shown in FIG. 9, shift start and end dates (defining a diagnosis period) can be determined and measured shifts in each of the plurality of physiological parameters during that diagnosis period can be measured.

Trend shifts for the measured physiological parameters of weight BP-diastolic, BP-systolic, pulse and gross motor activity (as displayed and described in FIG. 9) are then input into a fuzzy model to detect a change in patient condition and identify possible causes for the change. Shifts in weight (i.e., weight gain/loss) and blood pressure, along with variances in pulse can be analyzed by the fuzzy model and a change in patient condition can be identified.

Although the present invention has been shown and described with reference to preferred embodiments and examples thereof, it will be readily apparent to those of ordinary skill in the art that other embodiments and examples may perform similar functions and/or achieve similar results. For example, although mean shifts are measured and utilized herein, scatter shifts (amount and/or shape), the ratios of trend shifts, and/or the like may also be measured and utilized. Statistical techniques other than those specifically described may also be utilized.

A technical contribution for the disclosed method and apparatus is that is provides for a computer-implemented system and method for detecting a change in patient condition and identifying possible causes for the change based on trend shifts in physiological data.

Therefore, according to one embodiment of the invention, an automated method for diagnosing the cause of a trend shift in physiological data includes the step of receiving physiological data from a patient under observation, the physiological data comprising data on a plurality of measured physiological parameters. The method also includes the steps of performing a statistical analysis on a portion of the physiological data to determine a measured shift confidence interval in each of the plurality of physiological parameters and defining a signature shift for each of the plurality of physiological parameters, wherein the signature shifts for the plurality of physiological parameters are indicative of a pre-determined medical condition. The method further includes the steps of comparing the measured shift confidence interval of each of the plurality of physiological parameters to the signature shift associated with each of the plurality of physiological parameters and detecting a change in patient condition based on the comparison between the measured shift confidence interval and the signature shift of each of the plurality of physiological parameters.

According to another embodiment of the invention, a patient monitoring system includes a patient monitoring device configured to acquire physiological data from a patient under observation, the physiological data providing a measurement of at least one physiological parameter. The patient monitoring system also includes a computer in communication with the patient monitoring device to receive physiological data therefrom. The computer is programmed to receive physiological data from the monitoring device and select an analysis period that includes at least a portion of the acquired physiological data, the analysis period having a start date and an end date. The computer is further programmed to select data sets from the analysis period near the start date and the end date that have a predetermined size without violating normal scatter, measure a shift confidence interval between the data set near the start date and the data set near the end date using one or more statistical tests, and combine the shift confidence interval with a fuzzy model to achieve a physiological condition assessment, the fuzzy model describing how the mean associated with the at least one physiological parameter shifts when a predetermined physiological condition is present.

According to yet another embodiment of the invention, a computer readable storage medium includes thereon a computer program to provide a physiological condition assessment based on trend shifts in physiological data. The computer program comprises a set of instructions that, when executed by a computer, causes the computer to receive physiological data on a plurality of physiological parameters, determine a trend shift in the plurality of physiological parameters based on a statistical analysis of the physiological data, input the trend shift into a fuzzy model to identify a patient condition, and validate the fuzzy model using a plurality of validation cases comprising examples of diagnosed medical conditions determined from known shifts in the plurality of physiological parameters. The set of instructions further causes the computer to use an evaluation function to determine how well the fuzzy model differentiates the identified patient condition from a plurality of incorrect patient conditions for the plurality of validation cases.

While the invention has been described in detail in connection with only a limited number of embodiments, it should be readily understood that the invention is not limited to such disclosed embodiments. Rather, the invention can be modified to incorporate any number of variations, alterations, substitutions or equivalent arrangements not heretofore described, but which are commensurate with the spirit and scope of the invention. Additionally, while various embodiments of the invention have been described, it is to be understood that aspects of the invention may include only some of the described embodiments. Accordingly, the invention is not to be seen as limited by the foregoing description, but is only limited by the scope of the appended claims.

What is claimed is:

1. An automated method for diagnosing the cause of a trend shift in physiological data including the steps of:
receiving physiological data from a patient under observation, the physiological data comprising data on a plurality of measured physiological parameters;
performing a statistical analysis on a portion of the physiological data to determine a measured shift confidence interval in each of the plurality of physiological parameters;
defining a signature shift for each of the plurality of physiological parameters, the signature shifts for the plurality of physiological parameters indicative of a pre-determined medical condition;
comparing the measured shift confidence interval of each of the plurality of physiological parameters to the signature shift associated with each of the plurality of physiological parameters; and
detecting a change in patient condition based on the comparison between the measured shift confidence interval and the signature shift of each of the plurality of physiological parameters.

2. The method of claim 1 wherein the step of defining a signature shift includes selecting a fuzzy model comprised of a plurality of rules, the plurality of rules associating a shift in one of the plurality of physiological parameters to a shift in at least one other physiological parameter in the plurality of physiological parameters to define a pre-determined physiological condition.

3. The method of claim 2 wherein the step of comparing includes comparing the measured shift confidence interval of each of the plurality of physiological parameters to each of the plurality of rules.

4. The method of claim 2 wherein the step of detecting a change in patient condition includes determining if one of the plurality of rules matches the measured shift confidence intervals, wherein a rule matches if the measured shift confidence intervals of the physiological parameters match the signature shift for each physiological parameter.

5. The method of claim 4 further comprising the step of determining a confidence level in a match between the signature shifts and the measured shift confidence intervals.

6. The method of claim 5 wherein the step of determining a confidence level comprises providing an evaluation function that determines how well each of the plurality of rules and the signature shifts associated with each of those rules matches the measured shift confidence intervals.

7. The method of claim 5 further comprising the step of generating an alert when the confidence level meets a predetermined confidence threshold.

8. A patient monitoring system comprising:
a patient monitoring device configured to acquire physiological data from a patient under observation, the physiological data providing a measurement of at least one physiological parameter; and
a computer in communication with the patient monitoring device to receive physiological data therefrom, the computer programmed to:
receive physiological data from the monitoring device;
select an analysis period that includes at least a portion of the received physiological data, the analysis period having a start date and an end date;
select data sets from the analysis period near the start date and the end date that have a predetermined size without violating normal scatter;
measure a shift confidence interval between the data set near the start date and the data set near the end date using one or more statistical tests; and
combine the shift confidence interval with a fuzzy model to achieve a physiological condition assessment, the fuzzy model describing how the mean associated with the at least one physiological parameter shifts when a predetermined physiological condition is present.

9. The patient monitoring system of claim 8 wherein the computer is further programmed to remove outlying data points from the analysis period physiological data falling outside either an upper or a lower control limit.

10. The patient monitoring system of claim 8 wherein the one or more statistical tests comprise a two-sample t-test.

11. The patient monitoring system of claim 8 wherein the computer is further programmed to provide a plurality of validation cases associated with the system, the validation cases comprising examples of predetermined physiological conditions determined from known shifts in the at least one physiological parameter.

12. The patient monitoring system of claim 11 wherein the computer is further programmed to validate the fuzzy model using the plurality of validation cases.

13. The patient monitoring system of claim 11 wherein the computer is further programmed to provide an evaluation function.

14. The patient monitoring system of claim 13 wherein the computer is further programmed to use the evaluation function to determine how well the fuzzy model differentiates the physiological condition assessment from a plurality of incorrect physiological condition assessments for the plurality of validation cases.

15. The patient monitoring system of claim 14 wherein the computer is further programmed to output a plurality of confidence values for the physiological condition assessment and the plurality of incorrect physiological condition assessments.

16. The patient monitoring system of claim 15 wherein the computer is further programmed to generate an alert when the confidence value for the physiological condition assessment meets a predetermined confidence threshold.

17. The patient monitoring system of claim 15 wherein the computer is further programmed to determine the degree of separation between the physiological condition assessment and the plurality of incorrect physiological condition assessments.

18. The patient monitoring system of claim 17 wherein the computer is further programmed to optimize the degree of separation between the physiological condition assessment and the plurality of incorrect physiological condition assessments by randomly varying the fuzzy model within predetermined guidelines and recalculating the evaluation function.

19. The patient monitoring system of claim 17 wherein the computer is further programmed to select an analysis period based on one of an operator input and an identification of corner points in the received physiological data that provide a largest shift between the data set near the start date and the data set.

20. A computer readable storage medium having a computer program to provide a physiological condition assessment based on trend shifts in physiological data, the computer program comprising a set of instructions that when executed by a computer cause the computer to:
receive physiological data on a plurality of physiological parameters;
determine a trend shift in the plurality of physiological parameters based on a statistical analysis of the physiological data;
input the trend shift into a fuzzy model to identify a patient condition;
validate the fuzzy model using a plurality of validation cases, the validation cases comprising examples of predetermined patient conditions determined from known shifts in the plurality of physiological parameters; and
use an evaluation function to determine how well the fuzzy model differentiates the identified patient condition from a plurality of incorrect patient conditions for the plurality of validation cases.

21. The computer readable storage medium of claim 20 wherein the set of instructions further causes the computer to perform a two-sample t-test on the physiological data to determine a mean shift in the at least one physiological parameter.

22. The computer readable storage medium of claim 20 wherein the set of instructions further causes the computer to output a plurality of confidence values for the identified patient condition and the plurality of incorrect patient conditions.

23. The computer readable storage medium of claim 20 wherein the set of instructions further causes the computer to:
determine the degree of separation between the identified patient condition and the plurality of incorrect patient conditions; and
optimize the degree of separation between the identified patient condition and the plurality of incorrect patient conditions by randomly varying the fuzzy model within predetermined guidelines and recalculating the evaluation function.
